(19) United States
(12) Patent Application Publication
(10) Pub. No.: US 2024/0091053 A1
(43) Pub. Date: Mar. 21, 2024

(54) SYSTEMS AND METHODS FOR TEMPERATURE CONTROL IN DELIVERY OF THERMAL LIQUID TREATMENT

(71) Applicant: INTUITIVE SURGICAL OPERATIONS, INC., Sunnyvale, CA (US)

(72) Inventors: Robert L. Barry, Kirkland, WA (US); Erik M. Henne, Seattle, WA (US)

(21) Appl. No.: 18/259,051

(22) PCT Filed: Dec. 20, 2021
(86) PCT No.: PCT/US2021/064411

§ 371 Date: Jun. 22, 2023

Related U.S. Application Data
(60) Provisional application No. 63/130,149, filed on Dec. 23, 2020.

(51) Int. Cl. A61F 7/00; A61M 5/142; A61M 25/10

(52) U.S. Cl. CPC A61F 7/0085; A61M 5/14216; A61M 25/1018; A61F 2007/0054; A61F 2007/0056; A61M 2205/3368

(57) ABSTRACT

A heated liquid system may comprise a catheter including a liquid delivery channel for delivery of a treatment liquid from a distal end portion of the catheter. The catheter may also include a circulation supply channel extending along the liquid delivery channel. The circulation supply channel may be configured to convey a heated liquid from a heated liquid source toward the distal end portion of the catheter. The catheter may also include a circulation return channel extending along the liquid delivery channel. The circulation return channel may be configured to convey the heated liquid from the distal end portion toward a proximal end portion of the catheter. The heated liquid may be used to maintain a temperature of the treatment liquid.

### CROSS REFERENCE TO RELATED APPLICATIONS

This application claims the benefit of U.S. Provisional Application 63/130,149 filed Dec. 23, 2020, which is incorporated by reference herein in its entirety.

### FIELD

Examples described herein are related to systems and methods for endoluminal thermal treatment of diseased anatomy using a circulating heating system to maintain a temperature of a treatment liquid.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. In some examples, a minimally invasive medical instrument may be a thermal energy treatment instrument for use within an endoluminal passageway of a patient anatomy.

### SUMMARY

The following presents a simplified summary of various examples described herein and is not intended to identify key or critical elements or to delineate the scope of the claims.

In some examples, a heated liquid system may comprise a catheter including a liquid delivery channel for delivery of a treatment liquid from a distal end portion of the catheter. The catheter may also include a circulation supply channel extending along the liquid delivery channel. The circulation supply channel may be configured to convey a heated liquid from a heated liquid source toward the distal end portion of the catheter. The catheter may also include a circulation return channel extending along the liquid delivery channel. The circulation return channel may be configured to convey the heated liquid from the distal end portion toward a proximal end portion of the catheter. The heated liquid may be used to maintain a temperature of the treatment liquid.

In some examples, a method provides a heated treatment liquid through a catheter. The method may comprise circulating a heated circulation liquid through a circulation supply channel and a circulation return channel within the catheter. The method may also comprise delivering a heated treatment liquid through a liquid delivery channel within the catheter and through a distal end of the liquid delivery channel. The method may also comprise circulating a cooled circulation liquid through the circulation supply channel to the circulation return channel after delivering the heated treatment liquid through the distal end of the liquid delivery channel.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a patient anatomy according to some examples.

FIG. 2 is a cross-sectional view of a portion of an instrument illustrated in FIG. 1 according to some examples.

FIG. 3 is a schematic illustration of a medical system according to some examples.

FIGS. 4A and 4B illustrate liquid sources that may be used with the medical system of FIG. 3, according to some examples.

FIG. 5A illustrates a distal portion of a catheter with a heated liquid circulation system, according to some examples.

FIG. 5B illustrates a distal portion of a catheter with a heated liquid circulation system, according to some examples.

FIG. 5C illustrates a distal portion of a catheter with a heated liquid circulation system, according to some examples.

FIG. 6A illustrates a cross-sectional view of a catheter with a heated liquid circulation system, according to some examples. FIG. 6B illustrates a cross-sectional view of the catheter of FIG. 6A from a plane orthogonal to the plane of FIG. 6A.

FIG. 7A illustrates a cross-sectional view of a catheter with a heated liquid circulation system, according to some examples. FIG. 7B illustrates a cross-sectional view of the catheter of FIG. 7A from a plane orthogonal to the plane of FIG. 7A.

FIG. 8A illustrates a cross-sectional view of a catheter with a heated liquid circulation system, according to some examples.

FIG. 8B illustrates a perspective view of the catheter of FIG. 8A, according to some examples.

FIGS. 9A-9B illustrate perspective views of a catheter with an occlusion device, according to some examples.

FIG. 10A illustrates a side view of catheter with a heated liquid circulation system, according to some examples.

FIGS. 10B-10F are cross-sectional views of the catheter of FIG. 10A, according to some examples.

FIG. 10G illustrates the catheter of FIG. 10A, according to some examples.

FIG. 10H illustrates a cross-sectional view of a distal end of the catheter of FIG. 10G, according to some examples.

FIG. 10I illustrates the catheter of FIG. 10A positioned within an anatomic passageway, according to some examples, according to some examples.

FIG. 11 is a flowchart illustrating a method for applying a thermal energy treatment to an endoluminal passageway to occlude an adjacent blood vessel, according to some examples.

FIG. 12 is robot-assisted medical system, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The technology described herein provides techniques and treatment systems for endoluminal thermal treatment of diseased tissue. Although the examples provided herein may refer to treatment of lung tissue and pulmonary disease, it is understood that the described technology may be used in treating artificially created lumens or any endoluminal passageway or cavity, including in a patient trachea, colon, intestines, stomach, liver, kidneys and kidney calices, brain, heart, circulatory system including vasculature, fistulas, and/or the like. In some examples, treatment described herein may be referred to as endobronchial thermal liquid treatment and may be used in procedures to treat lung tumors and/or chronic obstructive pulmonary disease (COPD) that may include one or more of a plurality of disease conditions including chronic bronchitis, emphysema, and bronchiectasis.

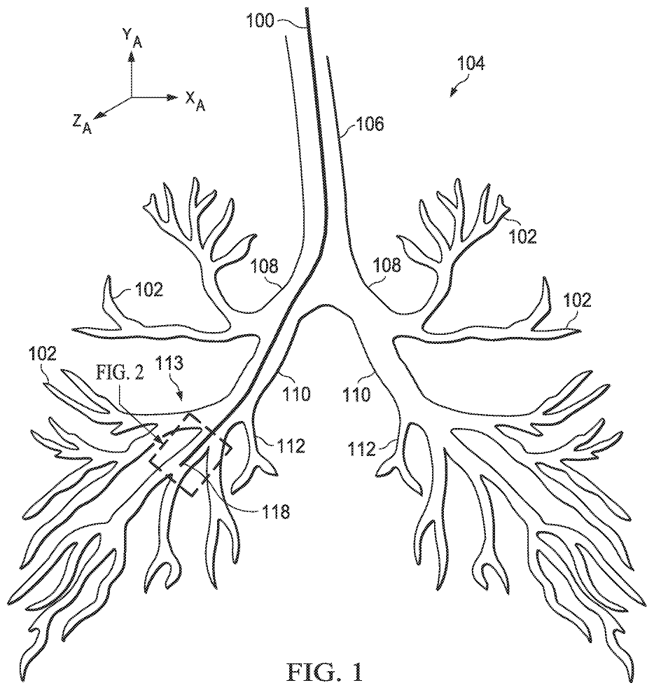

FIG. 1 illustrates an elongated medical instrument system 100 extending within branched anatomic passageways or airways 102 of an anatomical structure 104. In some examples the anatomic structure 104 may be a lung and the passageways 102 that include the trachea 106, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument 100 may be advanced into an anatomic opening (e.g. a patient mouth) and through the anatomic passageways 102 to perform a medical procedure, such as an endoluminal thermal energy treatment, at or near target tissue located in a region 113 of the anatomic structure 104.

FIG. 2 illustrates the distal end portion 118 of the elongated medical instrument system 100. A flexible catheter 150 includes an outer wall 152 surrounding an inner channel 153. A liquid circulation system 160 and a liquid delivery channel 162 extend within the inner channel 153 bounded by the outer wall 152. The liquid delivery channel 162 may have a channel wall 163 and may carry a heated treatment liquid 154 from a proximal end portion of the catheter 150, through the distal end portion 118, and may deliver the heated treatment liquid 154 to an area 156 distal of a distal opening 158 of the liquid delivery channel 162. The liquid circulation system 160 may include a circulation supply channel 164 that carries a heated thermal control liquid 166 in a distal direction D and a circulation return channel 168 that carries the heated thermal control liquid 166 is a proximal direction P. The heated thermal control liquid 166 in the circulation supply and return channels 164, 168 flows adjacent to the treatment liquid 154 in the liquid delivery channel to insulate the liquid delivery channel 162 and maintain or control a temperature of the heated treatment liquid 154 along the length of the catheter 150. In one embodiment, the flow rate of the heated thermal control liquid may be higher than the flow rate of the heated treatment liquid to aid in maintaining the temperature of the heated treatment liquid. In this configuration, the heated thermal control liquid 166 may serve as an insulating jacket. Since the temperature of the circulation channels 164/168 are approximately the same as the delivery channel 162, there may be little or no heat transfer between the channels and therefore no heat loss from the delivery channel 162. There may be some heat loss from the circulation channels 164/168 to ambient, but the fluid flow circulates newly heated fluid and removes the cooler fluid.

In some examples, during a treatment procedure using the medical instrument system 100, the treatment liquid 154 may be a heated saline or gel used to provide a thermal treatment to the region 113 of the anatomic structure. In these examples, the thermal control liquid 166 may be a heated liquid that helps maintain the temperature of the treatment liquid 154 and may flow at a higher flow rate than the flow rate of the treatment liquid 154. In some examples, the treatment liquid 154 may enter the liquid delivery channel at a temperature of between approximately 50° C. and 99° C. and may be maintained at approximately the same temperature by the thermal control liquid which may enter the circulation supply channel at a temperature of between approximately 50° C. and 99° C. In some embodiments, the temperature of the treatment liquid and the heated thermal control liquid may enter the catheter at between approximately 95° C. and 99° C. In some embodiments, for liquids that have vaporization temperatures greater than 99° C., the temperature of the treatment and/or heated thermal control liquid may be greater than 99° C. In some embodiments, the flow rate of the thermal control liquid 166 may be three to four times faster than the flow rate of the treatment liquid. For example, the flow rate of the thermal control liquid 166 may be approximately 240 ml/min and the flow rate of the treatment liquid 154 may be approximately 70 ml/min. In other examples, the flow rate of the thermal control liquid 166 may be between approximately 20 to 100 ml/min and the flow rate of the treatment liquid 154 may be between approximately 0.2 to 1.0 ml/sec. In some examples, the flow rate of the thermal control liquid 166 may be between approximately 40 and 80 ml/min. In some alternatives, the temperature of the heated thermal control liquid may have a temperature higher than the treatment liquid, and the flow rate of the thermal control liquid may be reduced. The flow rates and/or temperatures may be chosen to maintain a desired temperature of the heated treatment liquid 154, based on a maximum heated thermal control liquid 166 temperature, on device configurations including lengths of channels 162/164/168, thicknesses of the walls of channels 162/164,168, catheter diameter, on a type of control liquid/treatment liquid, and/or on anatomical conditions such as temperature of anatomy, fluid flow within anatomical lumen, size of lumen or organ, etc. In some alternatives, a volume of heated thermal control liquid may have a volume of approximately 1 ml to 20 ml delivered over a period between approximately 1 and 60 seconds.

In some examples, conditions such as lung cancer and emphysema may be treated with a treatment liquid at a temperature of approximately 95° C. at a flow rate of approximately 1 ml/sec. To achieve a treatment temperature of 95° C., an initial temperature at a proximal end may be approximately 97° C., to compensate for a small amount of heat loss that may occur despite the use of the heated circulation fluid. In some examples, conditions such as bronchitis may be treated with a treatment liquid at a temperature of approximately 57-63° C. In some examples, conditions such as bronchiectasis may be treated with a treatment liquid at a temperature of approximately 63-75° C.

After delivery of the treatment liquid 154, a cooled thermal control liquid may be circulated through the liquid circulation system to reduce the temperature of the liquid delivery channel 162 and the catheter 150 to avoid damage to adjacent patient tissue or damage to adjacent components (e.g., sensors, electronics, imaging components) that may be caused by prolonged exposure to heat. The cooled thermal control liquid may be at a room temperature or may have a controlled temperature between, for example, approximately 1° C. and 49° C. In some examples the temperature of the outer wall of the catheter may be controlled at a safety temperature (e.g., approximately 70° C.) by circulating the cooled thermal control liquid after the treatment liquid is delivered and/or by limiting the duration of the flow of the heated treatment liquid and the duration of the flow of the heated thermal control liquid to avoid generating a temperature of the outer catheter wall that exceeds the safety temperature.

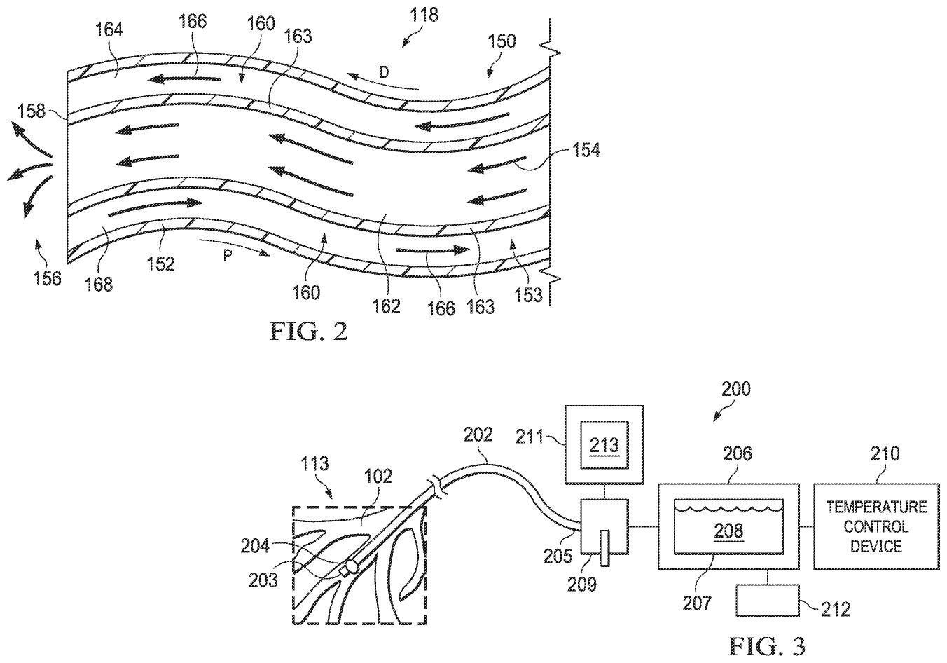

FIG. 3 illustrates a medical instrument system 200. The medical instrument system 200 may be, for example, the medical instrument system 100 and may include a catheter 202 with an occlusion device 204 coupled to a distal end portion 203 of the catheter 202. The catheter 202 may be similar to catheter 150 and may, in some embodiments, be inserted through an outer sheath (not shown). In some examples the catheter 202 and/or the sheath may be manually or robotically actuated or delivered using a robotically actuated elongate device. The occlusion device 204 may expand within a passageway 102 to prevent the flow of treatment liquid released from the distal end portion 203 of the catheter 202 proximally into the passageway 102. The occlusion device 204 may be, for example, an inflatable device such as a balloon fillable with an inflation medium such as air, saline, or another type of suitable fluid for expanding the balloon. The catheter 202 may be coupled to and in fluid communication with a fluid source 211 including a reservoir 213 that contains the inflation medium. In some examples, the proximal end portion 205 of the catheter 202 may be connected to the fluid source 211 via a control valve 209. In some embodiments the fluid source 211 may be a syringe including a fluid reservoir for containing a predetermined amount of inflation medium that may be injected into the occlusion device 204 to inflate the occlusion device. In some embodiments, for example, a 1 cm balloon occlusion device may be inflated with 1 cc of air from the syringe inflation device.

A proximal end portion 205 of the catheter 202 may be coupled to and in fluid communication with a fluid source 206 including a reservoir 207 that contains a non-compressible fluid 208, such as a liquid. In some examples, the proximal end portion 205 may be coupled to the fluid source 206 via the control valve 209 or a separate control valve. The temperature of the liquid 208 may be maintained by a temperature control device 210. The temperature control device 210 may include a heating system for heating the liquid 208. The heating system may include a heat generator, a temperature sensor, and other temperature regulation and generation components. In some examples, the heating system may heat the liquid 208 in the reservoir 207 with resistive heating, radiofrequency heating, ultrasonic heating, laser heating, magnetic heating, and/or microwave heating.

In some examples, the heated liquid 208 may be used as both the treatment liquid (e.g., treatment liquid 154) and the thermal control liquid (e.g., the thermal control liquid 166), and thus the fluid reservoir 207 may be in fluid communication with a liquid circulation system (e.g. liquid circulation system 160) and a liquid delivery channel (e.g., liquid delivery channel 162) of the catheter 202. In some examples, the temperature control device 210 may heat the treatment liquid to a temperature of less than a vaporization temperature for the treatment liquid. The liquid 208 may be, for example, water, saline, gel, glycerin, solution, or oil that maintains a liquid state at temperatures approaching 100 degrees Celsius. Depending on the components of the liquid, it may be heated to a temperature greater than 100 degrees Celsius while maintaining a liquid state. Glycerin and oil-based liquids may, for example, have boiling points greater than 100 degrees Celsius and thus may be used at temperatures higher than 100 degrees Celsius. In some examples, the liquid may be heated to a temperature between approximately 50 and 200 degrees Celsius. The liquid 208 may include any of the liquid materials or additives described in other embodiments.

An optional pressurization system 212 may be coupled to the reservoir 207 to pressurize the liquid 208 and urge the liquid 208 into the catheter 202 and through the liquid circulation system. The pressurization system 212 may pressurize the liquid using, for example, a linear actuator, a screw pump, a piston pump, a rotary pump, a diaphragm pump, or a peristaltic pump. In some examples, the reservoir 207 may be a syringe and may be heated to approximately 98° C. by the temperature control device 210. In some examples, the liquid 208 may be pressurized by heating.

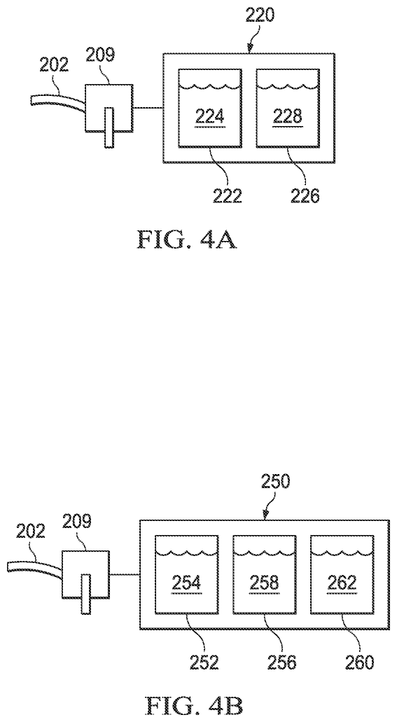

In some examples, as shown in FIG. 4A, a fluid source 220 may be coupled to the catheter 202 and may include a reservoir 222 for containing and maintaining a temperature of a liquid 224 and a reservoir 226 for containing and maintaining a temperature of a liquid 228. In some examples the liquid 224 may be a heated liquid used as the treatment liquid 154 and the heated thermal control liquid 166, and the liquid 228 may be a cooling liquid used as cooling thermal control liquid 166. In these examples, the temperature control device 210 may also include a heating system for heating the treatment liquid and the heated thermal control liquid and a cooling system for cooling the cooling thermal control liquid. As described in greater detail below, the heated thermal control liquid may circulate with the delivery of the treatment liquid, and the cooling thermal control liquid may circulate after the treatment liquid is delivered to reduce the temperature of the catheter and prevent injury to adjacent tissue or instrument components.

In some examples, as shown in FIG. 4B, a fluid source 250 may be coupled to the catheter 202 and may include a reservoir 252 for containing and maintaining a temperature of a liquid 254, a reservoir 256 for containing and maintaining a temperature of a liquid 258, and a reservoir 260 for containing and maintaining a temperature of a liquid 262. In some examples the liquid 254 may be a heated liquid used as the treatment liquid 154, the liquid 228 may a heated liquid uses as the thermal control liquid 166, and the liquid 260 may be a cooling liquid used as cooling thermal control liquid 166. Thus, in this embodiment, the treatment liquid and the thermal control liquid may be maintained at different temperatures.

In some embodiments, dedicated valves may be used with any or all of the fluid sources or reservoirs in the medical instrument system. In some embodiments, one or more multi-way valves may be used to control the flow of any or all of the fluid sources. In some embodiments, dedicated pumps, valves, or other flow control mechanisms may be used to provide dedicated control of the activation and speed of flow of fluids from each of the fluids in a fluid source or reservoir. For example, the flow of the thermal control liquid may be controlled at a faster rate than the flow of the treatment liquid. In some embodiments, the temperature, flow rate, flow initiation, flow termination, or other control aspects of the liquid circulation system of liquid delivery may be controlled by a robot-assisted medical system. In some examples, separate pumps may be placed in-line with the separate fluid reservoirs to control different fluid flow rates.

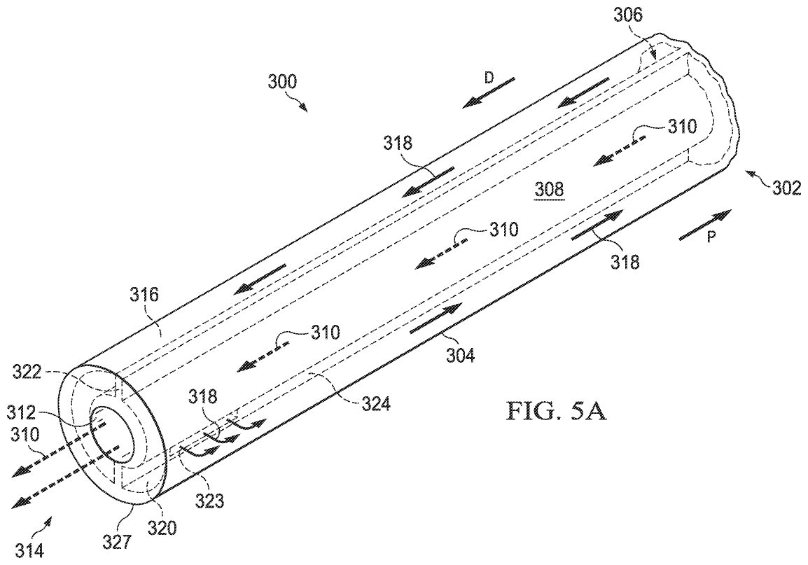

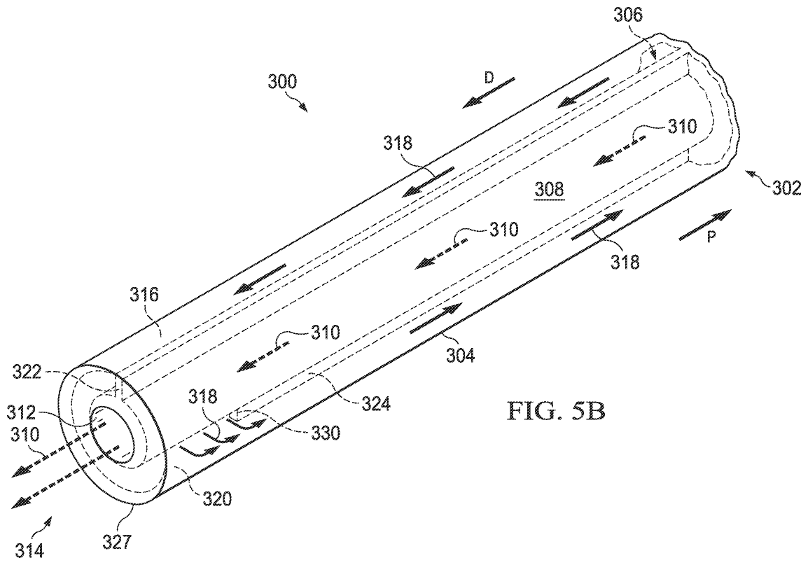

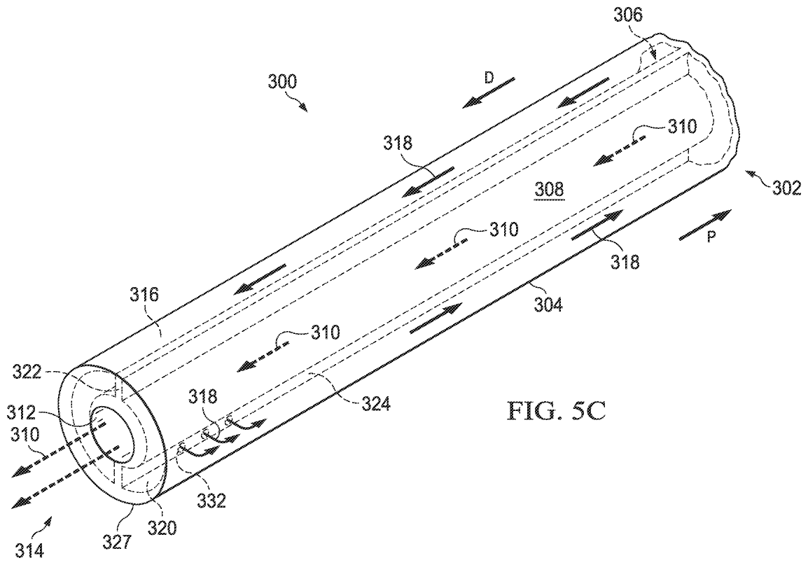

FIG. 5A illustrates a distal portion of a catheter 300 (e.g., the catheter 150, 202) with a liquid circulation system 302. The catheter 300 includes an outer wall 304 surrounding an inner channel 306. A liquid delivery channel 308 extends within the inner channel 306. The liquid delivery channel 308 may carry a treatment liquid 310 to a distal opening 312 for release to an area 314 distal of the catheter 300. The liquid circulation system 302 may include a circulation supply channel 316 that carries a thermal control liquid 318 in a distal direction D and a circulation return channel 320 that carries the thermal control liquid 318 is a proximal direction P. In this example, the circulation supply channel 316 and the circulation return channel 320 have generally C-shaped cross-sections with each channel 316, 320 surrounding approximately half of the liquid delivery channel 308. The circulation supply channel 316 and the circulation return channel 320 may each be sealed at the distal end of the catheter 300. In this example, an upper septum 322 and a lower septum 324 separate the adjacent channels 316, 320. A distal portion of the lower septum 324 may include a slot 323 that allows through passage of the thermal control liquid 318 from the circulation supply channel 316 to the circulation return channel 320. In other words, the thermal control liquid 318 may flow through the circulation supply channel 316 in the distal direction D, through the slot 323, and return through the circulation return channel 320 in the direction P. In some alternatives, a slot 323 may be formed in both the upper septum 322 and the lower septum 324 to allow additional circulation of the thermal control liquid 318 from the circulation supply channel 316 to the circulation return channel.

In some alternatives, as shown in FIG. 5B, the septum 324 may terminate proximally of the distal end 327 of the catheter thus forming a distal notched opening 330 through which the thermal control liquid 318 may flow from the circulation supply channel 316 to the circulation return channel 320. In some alternatives, the upper septum 322 may also or alternatively include a notched opening. In some alternatives, a plurality of notched openings may extend through the septum(s).

In some alternatives, as shown in FIG. 5C, septum 324 may include other types of through passages such as perforations 332 or holes for allowing circulation of the thermal control liquid 318 from the circulation supply channel 316 to the circulation return channel 320. In some alternatives, the upper septum 322 may also or alternatively include perforations.

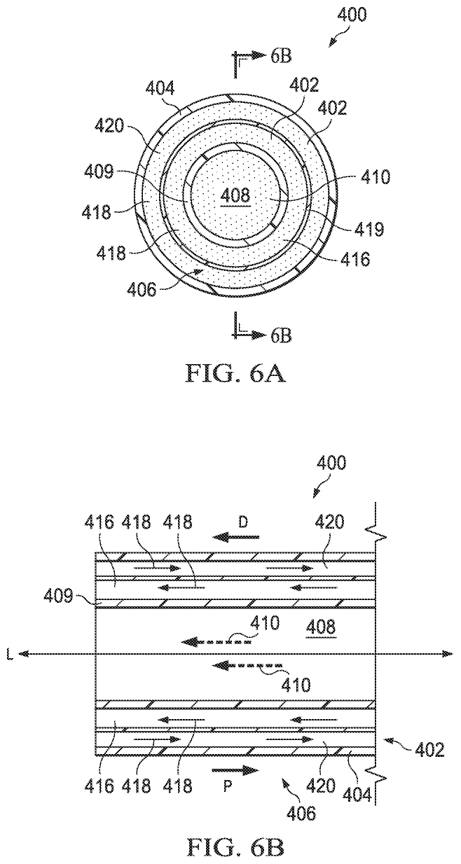

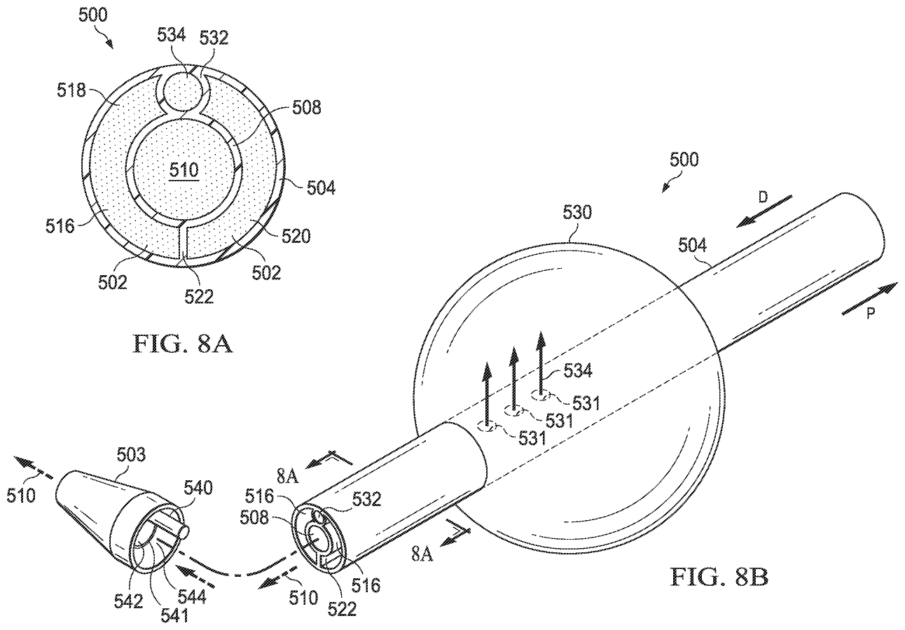

FIG. 6A illustrates a cross-sectional view of a catheter 400 with a heated liquid circulation system 402. The catheter 400 may be used, for example, in a medical system 200. FIG. 6B illustrates a cross-sectional view of the catheter 400 of FIG. 6A. The catheter 400 includes an outer wall 404 surrounding an inner channel 406. A liquid delivery channel 408, having a channel wall 409, extends within the inner channel 406. The liquid delivery channel 408 may carry a treatment liquid 410 through the catheter 400. The liquid circulation system 402 may include a circulation supply channel 416 that carries a thermal control liquid 418 in a distal direction D and a circulation return channel 420 that carries the thermal control liquid 418 is a proximal direction P. In this example, the circulation supply channel 416 and the circulation return channel 420 may be separated by a wall 419 and may be generally concentric with each other and with the liquid delivery channel 408. In this example, the channels 408, 416, 420 may be concentric about a longitudinal axis L but in other embodiments may be concentric about a different axis. In some examples, the thermal control liquid 418 may flow from the circulation supply channel 416 to the circulation return channel 420 through a distally connected connection chamber as described below in FIG. 8B. In some examples, the thermal control liquid 418 may flow through distal slots, notched openings, perforations or other apertures in the wall 419.

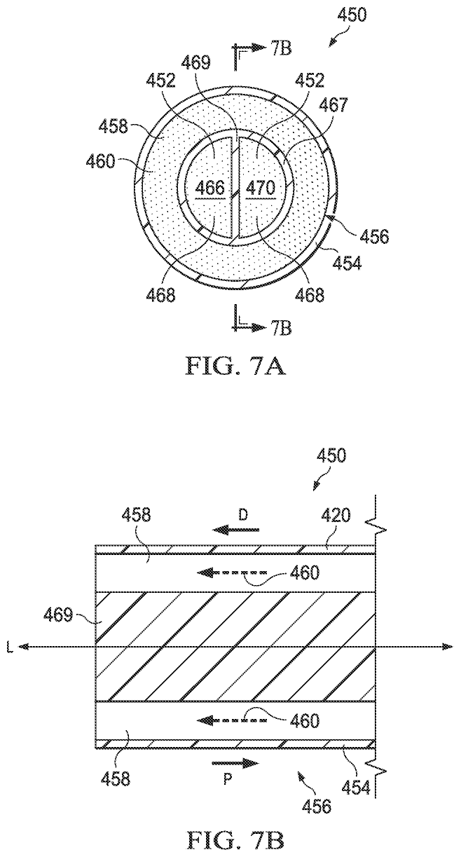

FIG. 7A illustrates a cross-sectional view of a catheter 450 with a heated liquid circulation system 452. The catheter 450 may be used, for example, in a medical system 200. FIG. 7B illustrates a cross-sectional view of the catheter 450 of FIG. 7A. The catheter 450 includes an outer wall 454 surrounding an inner channel 456. A liquid delivery channel 458 extends within the inner channel 456. The liquid delivery channel 458 may carry a treatment liquid 460 through the catheter 450. The liquid circulation system 452 may include a circulation supply channel 466 that carries a thermal control liquid 468 in a distal direction D and a circulation return channel 470 that carries the thermal control liquid 468 is a proximal direction P. In this example, the circulation supply channel 466 and the circulation return channel 470 may be surrounded by a wall 467 separated by a septum or wall 469. In some examples, the thermal control liquid 468 may flow from the circulation supply channel 466 to the circulation return channel 470 through a distally connected connection chamber as described below in FIG. 8B. In some examples, the thermal control liquid 468 may flow through distal slots, notched openings, perforations or other apertures in the wall 469.

FIG. 8A illustrates a cross-sectional view of a catheter 500 with a heated liquid circulation system 502. The catheter 500 may be used, for example, in a medical system 200. FIG. 8B illustrates a perspective view of the catheter 500 with a distal cap 503. The catheter 500 includes an outer wall 504, and a liquid delivery channel 508 extends through the catheter. The liquid delivery channel 508 may carry a treatment liquid 510. The liquid circulation system 502 may include a circulation supply channel 516 that carries a thermal control liquid 518 in a distal direction D and a circulation return channel 520 that carries the thermal control liquid 518 is a proximal direction P. In this example, a lower septum 522 separates the lower adjacent ends of the channels 516, 520.

In this example, an occlusion device 530 may be coupled to and surround a portion of the outer wall 504. An occlusion delivery channel 532 may extend along the length of the catheter 500 to carry an inflation medium 534 for expanding the occlusion device 530 from a collapsed configuration to an expanded configuration (as shown in FIG. 8B). The inflation medium 534 may flow from the liquid delivery channel 508 into the occlusion device 530 through one or more lumen 531 When the occlusion device 530 is expanded in an anatomic passageway, proximal flow of the treatment liquid 510 released from the liquid delivery channel 508 may be limited or stopped by the occlusion device 530. The inflation medium may be, for example air, saline, or another type of suitable fluid.

In some examples, the outer wall 504 may have an outer diameter of approximately 0.103 inches and a wall thickness of approximately 0.005 inches, although smaller or larger dimensions may also be suitable. In some examples, the occlusion delivery channel 532 may have an inner diameter of approximately 0.019 inches, and the liquid delivery channel 508 may have an inner diameter of approximately 0.045 inches, although smaller or larger dimensions may also be suitable.

In this example, the distal cap 503 may be coupled to the distal end portion of the catheter 500. The distal cap 503 may include a plug member 540 that is sized to extend into the occlusion delivery channel 532 to prevent distal flow of the inflation medium 534. The distal cap 503 also includes a wall 541 forming a delivery channel 542 that aligns with the delivery channel 508 to provide passage for the treatment liquid 510 through a distal end of the cap 503 to an area for treatment. The distal cap 503 also includes a connection chamber 544 that at least partially surrounds the wall 541. When the distal cap 503 is coupled to the catheter 500, the thermal control liquid 518 may flow in the distal direction D from the circulation supply channel 516 and into the connection chamber 544. The connection chamber 544 may redirect the flow of the thermal control liquid 518 toward the circulation return channel 522 where it continues to flow in the proximal direction P.

The previously described configurations and shapes of liquid delivery channels and circulation channels provided are examples, and other arrangements, configurations, and shapes of channels that use a circulating heated liquid to maintain a temperature of a heated treatment liquid may also be suitable.

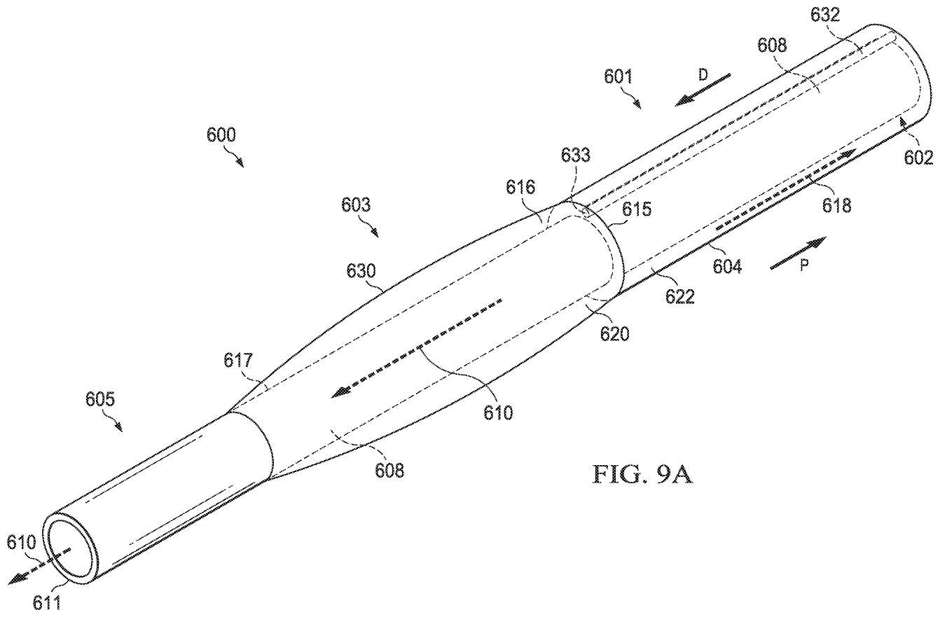

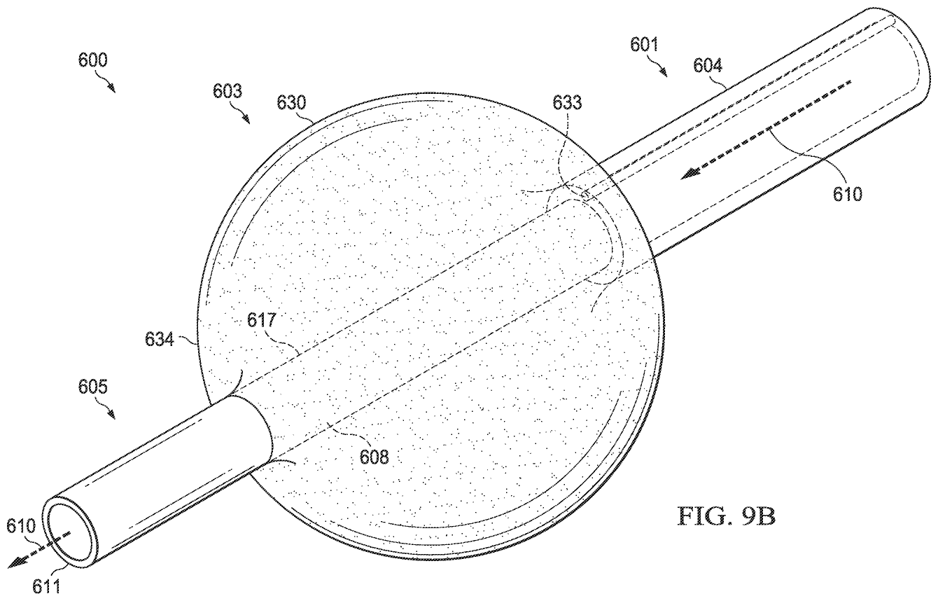

FIGS. 9A and 9B illustrate perspective views of a catheter 600 with an occlusion device 630 and a heated liquid circulation system 602. The catheter 600 may be used, for example, in a medical system 200. In FIG. 9A, the occlusion device 630 is collapsed, and in FIG. 9B, the occlusion device 630 is expanded. The catheter 600 includes an outer wall 604, and a liquid delivery channel 608 extends within the catheter 600. The liquid delivery channel 608 may carry a treatment liquid 610. The liquid circulation system 602 may include a circulation supply channel 616 that carries a thermal control liquid 618 in a distal direction D and a circulation return channel 620 that carries the thermal control liquid 618 in a proximal direction P. In this example, a lower septum 622 separates adjacent channels 616, 620.

In this example, the catheter 600 includes a proximal section 601, an intermediate section 603 and a distal section 605. The liquid circulation system 602 may extend within the proximal section 601, terminating at a distal end 615 of the outer wall 604. The liquid circulation system 602 may be substantially similar to any of the previously described liquid circulation systems, with fluid flowing from the circulation supply channel 616 to the circulation return channel 620 through the lower septum 622 as previously described. An occlusion delivery channel 632 may extend through the proximal section 601. The occlusion device 630 may be positioned along the intermediate section 603, coupled to the distal end 615 of the outer wall 604. A portion 617 of the liquid delivery channel 608 may extend through the occlusion device 630. The occlusion delivery channel 632 may carry an inflation medium 634 to the occlusion device 630 for expanding the occlusion device 630 from the collapsed configuration to the expanded configuration. A distal opening 633 of the occlusion delivery channel 632 may be surrounded by the occlusion device 630 such that the inflation medium 634 may flow from the occlusion delivery channel 632 into the occlusion device 630. In this example, the portion 617 of the liquid delivery channel 608 in the intermediate section 603 may have a smaller outer diameter than an outer diameter of the outer wall 604. Thus, the occlusion device 630 in the collapsed configuration may extend along the smaller outer diameter of the distal portion 617. The smaller outer diameter of the portion 617 of the liquid delivery channel 608 provides space to countersink the collapsed occlusion device 630. In some examples an outer diameter of the collapsed occlusion device 630 may be no larger than the outer diameter of the outer wall 604. The liquid delivery channel 608 may continue through to the distal opening 611 of the distal section 605. When the occlusion device 630 is expanded in an anatomic passageway, proximal flow of the treatment liquid 610 released from the liquid delivery channel 608 may be limited or stopped by the occlusion device 630. The inflation medium may be, for example air, saline, or another type of suitable fluid. In this example, because the circulation system 602 does not extend into the intermediate or distal sections 603, 605, the treatment liquid 610 may be delivered without insulation through those sections.

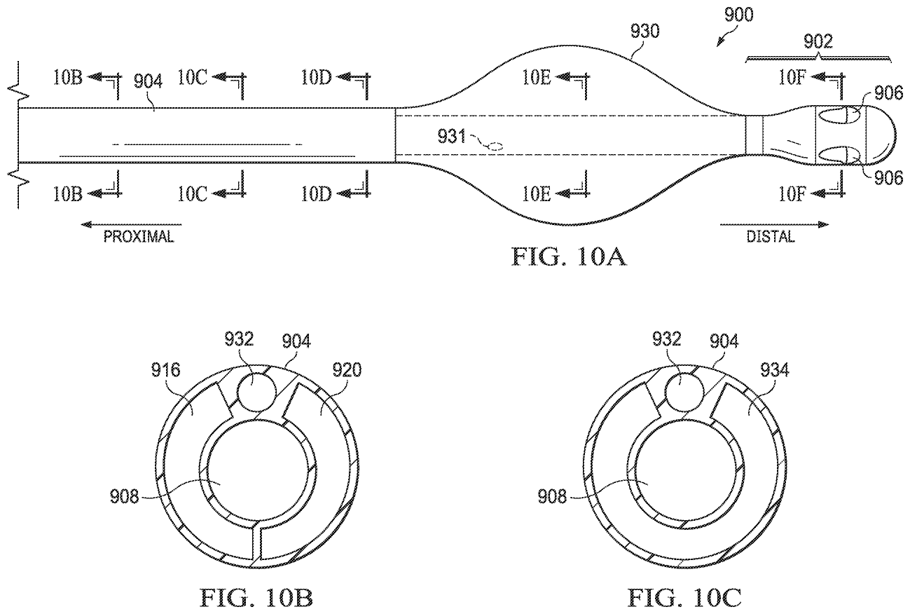

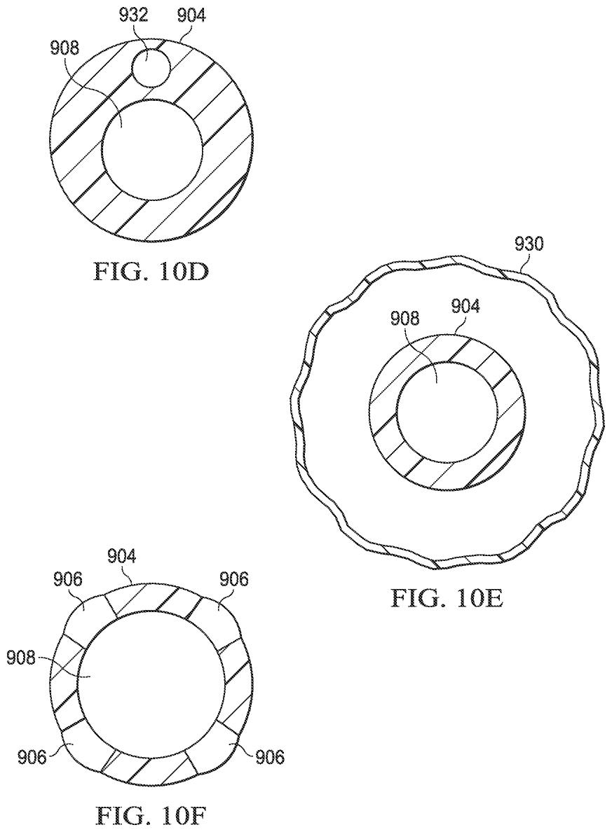

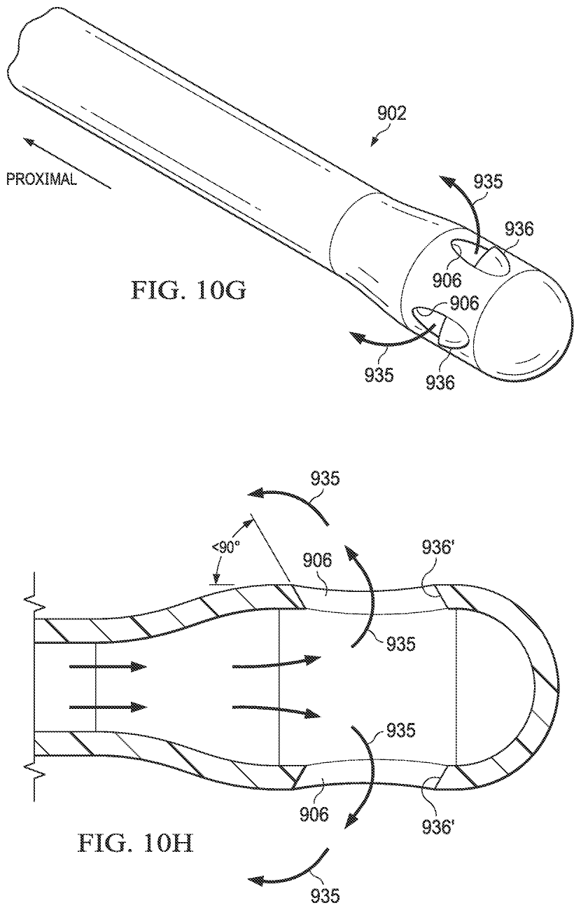

FIG. 10A illustrates a side view of a catheter 900 with an occlusion device 930 and a heated liquid circulation system. The catheter 900 may be used, for example, in a medical system 200. The catheter 900 includes an outer wall 904, and a liquid delivery channel 908 extends through the catheter. The liquid delivery channel 908 may carry a treatment liquid. The liquid circulation system may include a circulation supply channel 916 that carries a thermal control liquid in a distal direction and a circulation return channel 920 that carries the thermal control liquid in a proximal direction. In this example, the catheter 900 includes a distal portion 902 that extends distally of the occlusion device 930. One or more fluid diffusion ports 906 may extend through the outer wall 904 and may be in fluid communication with the liquid delivery channel 908. The ports 906 may direct the treatment liquid from the liquid delivery channel 908 in an initially proximal direction.

FIGS. 10B-10F provide cross-sectional views of the catheter 900 at a plurality of locations along the length of the catheter. As shown in FIG. 10B, at a location proximal of the occlusion device 930, septum 922 separates the lower adjacent ends of the channels 916, 920. At the cross-section of FIG. 10B, an occlusion delivery channel 932 may extend along the length of the catheter 900 to carry an inflation medium for expanding the occlusion device 930 from a collapsed configuration to an expanded configuration. In some examples, the outer wall 904 may have a diameter in a range of approximately 0.07-0.08 in. In some examples, the liquid delivery channel may have a diameter in a range of approximately 0.025-0.035 in. In some examples, the occlusion delivery channel may have a diameter in a range of approximately 0.008-0.012 in. The wall thickness may range between approximately 0.002 and 0.005 in.

As shown at the cross-section of FIG. 10C, a cross-flow channel 934 allows the thermal control liquid to flow from the circulation supply channel 916 to the circulation return channel 920. As shown at the cross-section of FIG. 10D, the circulation and cross-flow channels have terminated and the occlusion delivery channel 932 and liquid delivery channel 908 continue to extend through the catheter 900. As shown at the cross-section of FIG. 10E, the occlusion delivery channel 932 has terminated at or near a lumen 931 through which the inflation medium flows from the occlusion delivery channel into the occlusion device 930. At the distal portion 902 of the catheter 900, as shown in the cross-section of FIG. 10F, the ports 906 are in fluid communication with the liquid delivery channel 908 to dispense the treatment liquid. In the distal portion 902 the liquid delivery channel 908 may have a larger inner diameter and outer diameter as compared to more proximal portions. The larger diameter may reduce the pressure of the treatment liquid leaving the port 906. At the distal portion 902, the catheter 900 may be formed of a material that is softer than more proximal portions, to allow the ports 906 to be cut or formed. The distal end of the distal portion 902 may be closed to force the treatment liquid 935 from the catheter 900. As shown in FIG. 10G, fluid guides 936 may direct the treatment liquid 935 expelled from the port 906 in a proximal direction. In some examples, as shown in FIG. 10G, the fluid guide 936 may be an extension, a lip, or a hood. In other examples, as shown in FIG. 10H, the fluid guide 936′ may be an angled surface (a proximally directed, acute angle) of the outer wall 904 that directs the treatment liquid proximally along the outer wall 904. As shown in FIG. 10I, with the catheter 900 disposed in an anatomic lumen 938, the occlusion device 930 may be inflated to seal the anatomic lumen 938 and restrict proximal flow of treatment liquid 935. The heated liquid 935 may flow from the catheter 900 through the port 906 and in a proximal direction toward the occlusion device 930. The heated liquid 935, restricted by the occlusion device 930 may then wick distally along the anatomic wall, forming a fluid column in a distal flow direction along the anatomic wall. The fluid column may allow the heated liquid 935 to make a more complete, sustained, and therapeutic contact with the anatomic wall 937 of anatomic lumen 938, as compared examples in which the fluid is not initially directed in the proximal direction but instead forms a distal stream when exiting the lumen. Forcing the heated liquid 935 to exit in a proximal direction, rather than distal or radially, may slow down the fluid particle velocity and create capillary action between the passageway wall and ports as the droplet forms between them, wicking the liquid along the passageway wall. In some examples, four ports 906 may be formed in the catheter 900 and radially arranged about the longitudinal axis, but in other examples more or fewer ports may be used. In some examples, multiple rows of ports may be formed in the catheter. In some examples the occlusion device 930 may have an inflated outer diameter of approximately 6 mm. In some examples, the distal portion 902 may have a length of approximately 5 mm and a diameter of approximately 1.9 mm. In some examples, the ports 906 may have a diameter of approximately 0.75 mm.

In some examples, a distance between the widest portion of the inflated occlusion device and and the distal tip of the catheter may be minimized to prevent fluid from migrating into unintended passageways. In some examples for treating a condition such as emphysema, the occlusion device may be positioned in airways ranging from fourth to sixth generation. In some examples for treating a condition such as lung cancer, the occlusion device may be positioned in airways ranging from fourth to eighth.

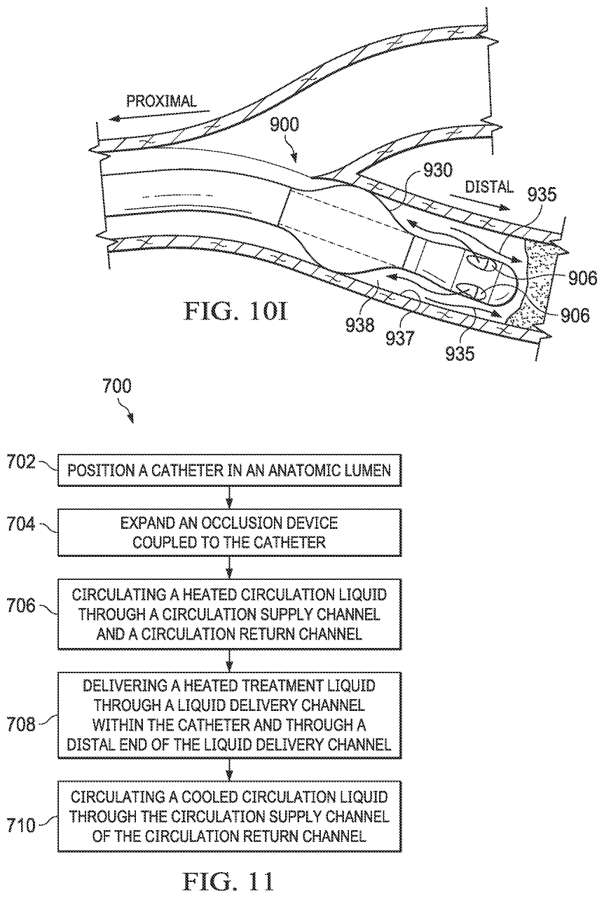

FIG. 11 is a flowchart illustrating a method 700 for applying a thermal energy treatment to an endoluminal passageway. The method 700 is illustrated as a set of operations or processes that may be performed in the same or in a different order than the order shown in FIG. 11. One or more of the illustrated processes may be omitted in some embodiments of the method. Additionally, one or more processes that are not expressly illustrated in FIG. 11 may be included before, after, in between, or as part of the illustrated processes. In some embodiments, one or more of the processes of method 700 may be implemented, at least in part, by a control system executing code stored on non-transitory, tangible, machine-readable media that when run by one or more processors (e.g., the processors of a control system) may cause the one or more processors to perform one or more of the processes.

At an optional process 702, a catheter of a medical instrument system, such as any of the catheters previously described, may be positioned in an anatomic passageway (e.g., a passageway 102). Pulmonary blood vessels or vasculature may extend alongside the bronchial passageway 102. A target tissue for treatment with the medical instrument system, which may be, for example a lung tumor, may be located distally of or downstream from the positioned distal end of the catheter. In some examples, the target tissue may be located throughout a region of the anatomy (e.g., region 113). The positioning of the catheter may be performed with a robot-assisted endoluminal medical system or may be performed with an endoscope manually by a clinician.

At an optional process 704, an occlusion device, such as any of the occlusion devices previously described, may be expanded in the anatomic passageway. The occlusion device may engage of the walls of the adjacent anatomic passageway, forming a seal that may prevent or restrict liquid from flowing between the occlusion device and the anatomic passageway. In some examples, the occlusion device may be an expandable device such as an inflatable balloon, an expandable membrane, or an expandable hood that extends circumferentially around the catheter. The occlusion device may have a collapsed configuration (e.g., FIG. 9A) which allows for insertion or retraction within the anatomic passageway. The occlusion device 156 may have a deployed configuration (e.g. FIGS. 8B, 9B) in which the occlusion device extends into contact with the wall of the anatomic passageway to form a seal or barrier preventing fluid flow proximally of the occlusion device. In some embodiments, the deployed configuration of the occlusion device may further function to lodge the catheter in the passageway to prevent translation of the catheter relative to the passageway. In some embodiments, the occlusion device may expand to a predetermined size and configuration or a predetermined pressure. In some embodiments, the extent of the expansion of the expandable device may be controlled based, for example, on the diameter of the passageway. In some embodiments, the location and extend of the expansion may be monitored by a visualization system. In some embodiments, the extent of the expansion may be monitored and/or controlled by a robot-assisted medical system control system (e.g. control system 812). For example, the robot-assisted medical system may receive sensor data from the visualization system or from an external imaging system indicating a diameter of a passageway or an extent of contact with the passageway and may cease expansion of the expandable device when an effective seal has been achieved.

At a process 706, a heated thermal control liquid (e.g., thermal control liquid 166, 318, 418, 468, 518) may be circulated through a circulation supply channel (e.g., circulation supply channel 164, 316, 416, 466, 516) in the catheter and to a circulation return channel (e.g., circulation supply channel 168, 320, 420, 470, 520) in the catheter. The thermal control liquid may be heated and contained, for example, in a reservoir (e.g. reservoir 207, 222, 256) at a temperature of between approximately 95° C. and 99° C. The thermal control liquid may be injected, pumped, or otherwise conveyed into and through the circulation supply channel. In some alternatives, the thermal control liquid may be heated to a temperature above 99° C. that is still below a vaporization temperature for the thermal control liquid.

At a process 708, a heated treatment liquid may be delivered through a liquid delivery channel (e.g., liquid delivery channel 162, 308, 408, 458, 508) within the catheter. The heated treatment liquid may be dispensed from the liquid delivery channel into the anatomic lumen. The heated treatment liquid may be heated and contained, for example, in a reservoir (e.g. reservoir 207, 222, 252) at a temperature of between approximately 95° C. and 99° C. The heated treatment liquid may be injected, pumped, or otherwise conveyed into the liquid delivery channel. In some embodiments, the temperature of the heated treatment liquid may be maintained at a target delivery temperature of between approximately 95° C. and 99° C. by the heated thermal control liquid while in transit along the liquid delivery channel. While the heated treatment liquid is in the liquid delivery channel of the catheter, the heated thermal control liquid may be circulating in adjacent circulation channels providing insulation and heating to maintain the temperature of the heated treatment liquid within an acceptable treatment range as it travels along the catheter. Without the circulating heated thermal control liquid, the temperature of the treatment liquid could drop during transit through the catheter to an unacceptable temperature for treatment in the anatomic passageway, with longer catheters experiencing greater drops in temperature. In some examples, the heated thermal control liquid in the circulation supply and return channels may have a flow rate of, for example, approximately 240 ml/min, although the flow rate may be greater or less than 240 ml/min. In those examples, the heated treatment liquid may have a flow rate of approximately 70 ml/min (although the flow rate may be greater or less than 70 ml/min) through the liquid delivery channel. The more rapid flow rate of the thermal control liquid as compared to the flow rate of the heated treatment liquid may prevent a temperature drop below the target delivery temperature. In some examples, the heated thermal control liquid may be circulated prior to the flow of the treatment liquid to pre-heat the liquid delivery channel.

In some embodiments, the released heated treatment liquid may directly contact the walls of the anatomic lumen causing ablation at and/or near the target tissue. In other embodiments, the released heated treatment liquid may flow into an expandable device such as a silicone balloon that may contain the heated treatment liquid but allow the transfer of heat to the adjacent tissue to ablate the tissue. In such examples, an occlusion balloon may be omitted. After the ablation with the heated balloon, the heated treatment liquid may be evacuated through the liquid delivery channel and may, in some examples, return to a fluid reservoir. Whether ablated by direct contact with the treatment liquid or by a balloon filled with the treatment liquid, the depth of ablation and therefore the anatomical structures (e.g., bronchial passageway, bronchial artery, pulmonary artery, etc.) occluded by the ablation may be controlled, for example, based on the amount of liquid released from the catheter and the temperature of the heated treatment liquid. Ablation may induce cellular and structural changes in the epithelium that in some cases may extend to the sub-epithelium. The ablation may cause tissue reduction, including destruction of goblet cells and cilia in lung tissue. In some embodiments, the cellular matrix may be preserved to allow for later regrowth of healthy cells. In some examples, the tissue reaction may occur entirely during the application of the heated treatment liquid, and in other examples, the tissue damage may develop over a period of time as the anatomy responds to the injury caused by the heat. A proximal flow of the heated treatment liquid in the anatomic lumen may be restricted by the occlusion device, thus urging the dispensed treatment liquid into an area of the anatomic passageway distal of the catheter.

In some embodiments, the catheter may be moved (e.g., retracted) during the delivery of the heated treatment liquid. In some embodiments, the movement may be performed manually. In some embodiments, the treatment device may be coupled to a manipulator of a robot-assisted medical system (e.g., a system 800) and movement of the treatment device from a first location to a second location may be performed by actuation of a manipulator. In some embodiments, the occlusion device can remain inflated during retraction or might need to be deflated slightly during retraction. The amount of deflation may, for example, be based on sensed pressure, be a predetermined delta from the inflated state, or be determined based on visual feedback (e.g., user determined or by image recognition).

If the circulating heated thermal control liquid and/or the heated treatment liquid raise the temperature on the outside surface of the outer wall of the catheter for an extended period of time, the catheter may damage the adjacent anatomic tissue. Thus, this treatment method may maintain the treatment temperature of the treatment liquid while maintaining an external temperature along the catheter than minimizes thermal risk to the adjacent tissue. At an optional process 710, after the heated treatment liquid has been dispensed into the anatomic passageway, a cooled thermal control liquid may be circulated through the circulation supply channel and the circulation return channel to cool the catheter and prevent damage to the adjacent patient anatomy or to other components such as sensors, electronics, or imaging components in the catheter or in a sheath through which the catheter extends.

In some examples, an outside temperature of the outer wall of the catheter may be maintained at a pre-determined safety temperature of, for example, 70° C. by controlling the duration of the flow of the heated treatment liquid and the duration of the flow of the heated thermal control liquid to prevent outer wall temperatures from exceeding the safety temperature. Temperature sensors may be included within or along the outer wall of the catheter to measure temperature, and the duration of flow may be altered based on the sensed temperature, in a closed loop manner. In some examples, the flow rate, flow duration, and/or fluid temperature may be altered based on temperature of the catheter wall. The temperature of the treatment fluid may be monitored (e.g., with a temperature sensor within the delivery fluid lumen) and may be used to adjust the temperature, flow rate, and/or duration of delivery of the circulating fluid. In some embodiments the temperature of the treatment fluid may be monitored along different lengths of the delivery fluid lumen, such as at a proximal location, a distal location immediately before fluid exit from the delivery channel, or multiple points in between to determine change in temperature as fluid is delivered down the length of the catheter. Additionally or alternatively, in some embodiments, the cooled thermal control liquid may circulate through the circulation supply and return channels, after delivery of the treatment liquid, to maintain the temperature of the outer wall of the catheter at or below the safety temperature.

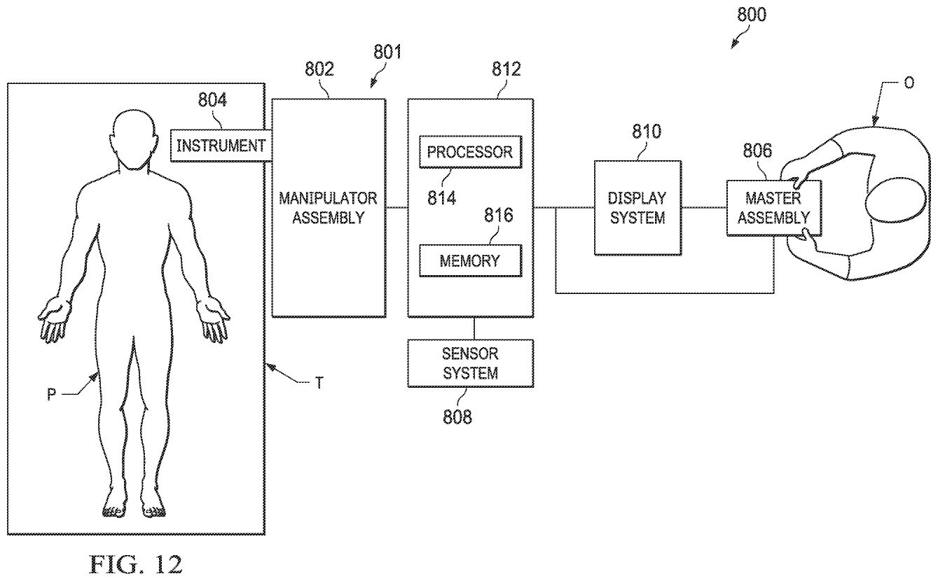

In some embodiments, the systems and methods disclosed herein may be used in a medical procedure performed with a robot-assisted medical system as described in further detail below. As shown in FIG. 12, a robot-assisted medical system 800 may include a manipulator assembly 802 for operating a medical instrument 804 (e.g., medical instrument system 100, 200, or any of the instruments described herein) in performing various procedures on a patient P positioned on a table T in a surgical environment 801. The manipulator assembly 802 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 806, which may be inside or outside of the surgical environment 801, generally includes one or more control devices for controlling manipulator assembly 802. Manipulator assembly 802 supports medical instrument 804 and may optionally include a plurality of actuators or motors that drive inputs on medical instrument 804 in response to commands from a control system 812. The actuators may optionally include drive systems that when coupled to medical instrument 804 may advance medical instrument 804 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 802 may support various other systems for irrigation, treatment, or other purposes. Such systems may include fluid systems (including, for example, reservoirs, heating/cooling elements, pumps, and valves), generators, lasers, interrogators, and ablation components.

Robot-assisted medical system 800 also includes a display system 810 for displaying an image or representation of the surgical site and medical instrument 804 generated by a sensor system 808 which may include an endoscopic imaging system. Display system 810 and master assembly 806 may be oriented so an operator O can control medical instrument 804 and master assembly 806 with the perception of telepresence. Any of the previously described graphical user interfaces may be displayable on a display system 810 and/or a display system of an independent planning workstation.

The sensor system 808 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 804. The sensor system 808 may also include temperature, pressure, force, or contact sensors or the like.

Robot-assisted medical system 800 may also include control system 812. Control system 812 includes at least one memory 816 and at least one computer processor 814 for effecting control between medical instrument 804, master assembly 806, sensor system 808, and display system 810. Control system 812 also includes programmed instructions (e.g., a non-transitory machine-readable medium storing the instructions) to implement a plurality of operating modes of the robot-assisted medical system including a navigation planning mode, a navigation mode, and/or a procedure mode. Control system 812 also includes programmed instructions (e.g., a non-transitory machine-readable medium storing the instructions) to implement some or all of the processes described in accordance with aspects disclosed herein, including, for example, expanding the expandable device, regulating the temperature of the heating system, regulating valves to control fluid delivery, controlling fluid flow rate, controlling insertion and retraction of the treatment instrument, controlling actuation of a distal end of the treatment instrument, receiving sensor information, altering signals based on the sensor information, selecting a treatment location, and/or determining a size to which the expandable device may be expanded.

Control system 812 may optionally further include a virtual visualization system to provide navigation assistance to operator O when controlling medical instrument 804 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 812 may use a pre-operative image to locate the target tissue (using vision imaging techniques and/or by receiving user input) and create a pre-operative plan, including an optimal first location for performing bronchial passageway and vasculature occlusion. The pre-operative plan may include, for example, a planned size to expand the expandable device, a treatment duration, a treatment temperature, and/or multiple deployment locations.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions. Not all the illustrated processes may be performed in all embodiments of the disclosed methods. Additionally, one or more processes that are not expressly illustrated in may be included before, after, in between, or as part of the illustrated processes. In some embodiments, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some embodiments are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in embodiments of this disclosure may be implemented in software to execute on a processor of a computer system such as control processing system. When implemented in software, the elements of the embodiments of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

### Examples

Example 1. A method for providing a heated treatment liquid through a catheter, the method comprising:
   circulating a heated circulation liquid through a circulation supply channel and a circulation return channel within the catheter; and
   delivering a heated treatment liquid through a liquid delivery channel within the catheter and through a distal end of the liquid delivery channel; and
   maintaining a temperature of the heated treatment liquid during the delivery through the liquid delivery channel and delivery through the distal end of the liquid delivery channel.
Example 2. The method of Example 1, wherein delivering the heated treatment liquid through the distal end of the liquid delivery channel includes directing the treatment liquid in a proximal direction.
Example 3. The method of Example 2, wherein the distal end of the liquid delivery channel includes a plurality of distal openings radially spaced to provide wicking of the heated treatment liquid after delivering the liquid through the distal end.
Example 4. The method of Example 1, further comprising:
   inflating an occlusion device to restrict flow of the treatment liquid proximal of the occlusion device, wherein the occlusion device is coupled to the catheter proximal to the distal end of the liquid delivery channel.
Example 5. The method of any of Examples 1-4, further comprising:
   circulating a cooled circulation liquid through the circulation supply channel to the circulation return channel after delivering the heated treatment liquid through the distal end of the liquid delivery channel.
Example 6. The method of any of Examples 1-4, further comprising maintaining a temperature of an outer surface of the catheter at approximately 70° C. or less.
Example 7. The method of any of Examples 1-4, wherein the heated circulation liquid has a temperature of between approximately 50° C. and 99° C. when entering the circulation supply channel.
Example 8. The method of any of Examples 1-4, wherein the heated treatment liquid has a temperature of between approximately 50° C. and 99° C. when entering the liquid delivery channel.
Example 9. The method of any of Examples 1-4, wherein a volume between approximately 1 ml and 20 ml the heated treatment liquid is delivered over a period between approximately 1 and 60 seconds.
Example 10. The method of any of Examples 1-4, wherein the cooled circulation liquid has a temperature of between approximately 1° C. and 49° C. when entering the circulation supply channel.
Example 11. The method of any of Examples 1-4, wherein the heated circulation liquid has a flow rate of between approximately 40 and 80 ml/min in the circulation supply channel.
Example 12. The method of any of Examples 1-4, wherein the heated treatment liquid has a flow rate of approximately 70 ml/min in the circulation supply channel.

1. A heated liquid system comprising:
a catheter including:
a liquid delivery channel for delivery of a treatment liquid from a distal end portion of the catheter;
a circulation supply channel extending along the liquid delivery channel, the circulation supply channel configured to convey a heated liquid from a heated liquid source toward the distal end portion of the catheter; and
a circulation return channel extending along the liquid delivery channel, the circulation return channel configured to convey the heated liquid from the distal end portion toward a proximal end portion of the catheter,
wherein the heated liquid maintains a temperature of the treatment liquid.

2. The system of claim 1, wherein the circulation supply channel is separated from the circulation return channel by a septum and a distal end of the circulation supply channel and a distal end of the circulation return channel are sealed.

3. The system of claim 2, wherein the septum terminates proximally of the distal ends of the circulation return channel and the circulation supply channel.

4. The system of claim 2, wherein the septum is perforated to allow flow of the heated liquid through the septum.

5. The system of claim 1, wherein the liquid delivery channel includes a distal opening and wherein the liquid delivery channel is configured to direct the treatment liquid to the distal opening.

6. The system of claim 5, wherein the distal opening includes a guide, wherein the guide includes a proximally angled surface.

7. The system of claim 5, wherein the distal opening is one of a plurality of distal openings, wherein the plurality of distal openings is radially arranged around the liquid delivery channel.

8. The system of claim 5, further comprising:
an occlusion device coupled to the catheter proximal to the distal opening, the occlusion device expandable with an inflation medium.

9. The system of claim 8, wherein the liquid delivery channel extends to a distal end of the catheter and the occlusion device is coupled to the catheter proximal to the distal end of the catheter and distal of the circulation supply channel and the circulation return channel.

10. The system of claim 8, wherein the catheter further comprises an inflation medium delivery channel coupled to the occlusion device.

11. (canceled)

12. The system of claim 1, wherein the circulation supply channel extends around at least a portion of the liquid delivery channel and the circulation return channel extends around at least a portion of the liquid delivery channel.

13. The system of claim 1, wherein the circulation supply channel and the circulation return channel are at least partially surrounded by the liquid delivery channel.

14. The system of claim 1, wherein the circulation supply channel and the circulation return channel are concentric.

15. The system of any of claim 1, wherein the circulation supply channel and the circulation return channel have C-shaped cross-sections.

16. (canceled)

17. The system of claim 1, further comprising the heated liquid source, wherein the heated liquid source includes a first reservoir coupled to the liquid delivery channel and a second reservoir coupled to the circulation supply channel and the circulation return channel.

18. The system of claim 1, further comprising the heated liquid source and further comprising a heating device coupled to the heated liquid source, the heating device configured to heat the treatment liquid to a temperature of less than a vaporization temperature for the treatment liquid.

19. (canceled)

20. The system of claim 1, further comprising a distal cap coupled to the distal end portion of the catheter, the distal cap including a connection chamber between the circulation supply channel and the circulation return channel.

21. (canceled)

22. The system of claim 1, further comprising a treatment balloon coupled to the distal end portion of the catheter and configured to receive the treatment liquid from the distal opening at the distal end portion of the catheter.

23-24. (canceled)

25. The system of claim 1, wherein the circulation supply channel is further configured to convey a cooling liquid from a cooling liquid source toward a distal end portion of the catheter.

26. (canceled)

27. The system of any of claim 1, further comprising:
a control system configured to:
receive a sensed temperature of the treatment liquid near the distal end portion of the catheter and
responsive to the sensed temperature, adjust a temperature or a flow rate of the heated liquid.

28-39. (canceled)
